(19) United States
(12) Patent Application Publication
(10) Pub. No.: US 2018/0042813 A1
(43) Pub. Date: Feb. 15, 2018

(54) SMART EQUIPMENT WITH BIDIRECTIONAL DIAGNOSIS AND THERAPY DEVICE

(71) Applicant: INNO HEALTH TECHNOLOGY CO., LTD., Taichung (TW)

(72) Inventor: Chih-Cheng Chiang, Taichung (TW)

(21) Appl. No.: 15/236,957

(22) Filed: Aug. 15, 2016

(51) Int. Cl. A61H 39/00; A61N 1/36; A61B 5/00; A61N 1/04

(52) U.S. Cl. CPC A61H 39/002; A61N 1/0492; A61N 1/36014; A61B 5/4836

(57) ABSTRACT

The present invention is a smart equipment with bidirectional diagnosis and therapy device, comprising a power supply unit used for providing each unit with required power, a high-voltage diagnosis and therapy unit provided for diagnosis and therapy as well as electronic acupuncture and sending back a diagnosis and therapy signal, an input and display unit provided for inputting operation commands and displaying related image, a wireless transmission unit provided for connecting to a cloud database wirelessly, a magnetic disk installed with program being loaded with application software for processing the diagnosis and therapy signal correspondingly, and a microprocessor used for processing related operation. Thereby, the present invention enables the user to manipulate the high-voltage diagnosis and therapy unit for diagnosis and therapy via the input and display unit according to suggestion from application software. Thus, correct diagnosis and therapy is allowed for the user to achieve the best effect.

### FIELD OF THE INVENTION

The present invention is related to a smart equipment, particularly to a smart equipment combined with electronic medical device.

### BACKGROUND OF THE INVENTION

Referring to FIGS. 1 and 2, there are shown schematic diagrams of acupoints on a hand and feet corresponding to viscera and organs of human body, in which reflex zones of acupoints 1 refer to acupuncture points, such as acupoint for sciatic nerve 2, acupoint for cold 3 and etc., for parts of human body. In accordance with the theory of traditional Chinese medicine, various acupuncture points are corresponded to various organs, and moreover, stimulating acupuncture points for parts of human body by acupuncture is capable of treating diseases correspondingly, which is proven and accepted by the public. Acupuncture, however, is an invasive therapy, and should not be performed without medical license. Moreover, the needles used for acupuncture are generally reused by multiple users. Although these needles are disinfected, infection may be caused if disinfection is not completely or contamination occurs during storage. In other words, the crisis during the process of acupuncture is primarily resulted from incomplete disinfection process. Acupuncture, however, is further considered as one of folk remedies. Thus, it is often found illegal acupuncture engaged privately, which leads to significant hindrance to public health.

Therefore, referring to Taiwan patent publication no. M505940, there is disclosed a connector of computer peripheral with function of high-voltage pulse, the connector comprising a housing, a high-voltage pulse module, a computer-connecting interface and an electrode-connecting interface, in which the computer-connecting interface is capable of being connected to a computer, while the electrode-connecting interface is provided for connecting to a peripheral having a high-voltage pule electrode. Thereby, the operational status of the high-voltage pulse module may be controlled by the computer, such that the high-voltage pulse is outputted from the high-voltage pulse electrode of the peripheral to be used for electronic acupuncture.

The computer described above may be a smart equipment, such as a smartphone, a tablet and etc. That is to say, the smartphone may be utilized for controlling the high-voltage pulse electrode to output the high-voltage pulse. The high-voltage pulse electrode is used as an electronic acupuncture appliance so as to perform acupuncture on acupuncture points for parts of human body, and is suitable for smartphones of various models. The operation interface is shown on the smartphone, such that the effects of convenience, storage space and carriage space savings are achieved for a user to perform electronic acupuncture at any time and any place without anxiety about sanitation. Moreover, the operation and display interface of electronic acupuncture may be integrally displayed on the touch screen of the smartphone. The location diagram of acupuncture points of human body and the textual guidance for acupuncture may be then shown on the touch screen of the smartphone, such that the user is allowed to manipulate acupuncture on the smartphone.

The device as described above, combined with a computer, such as a smartphone, may be used as a portable electronic acupuncture appliance, which produces the effect of electrotherapy of stimulating various acupuncture points of human body through the high-voltage pulse electrode. The conventional electronic acupuncture appliance, however, is used for therapy to be better late than never when pathological changes in the organs of human body have been found, but not for diagnosis and therapy for self-health care. Moreover, although the location diagram of acupuncture points of human body and the textual guidance of acupuncture are provided for reference, the problem of insufficient or excessive duration of acupuncture often occurs because it is generally incapable of determining the time duration, during which acupuncture should be performed, accurately for a non-professional user.

### SUMMARY OF THE INVENTION

Therefore, it is the main object of the present invention to provide a smart equipment with bidirectional diagnosis and therapy device, in which the smart equipment is combined with the bidirectional diagnosis and therapy device, while the smart equipment is loaded with application software such that a user is allowed to perform bidirectional diagnosis and therapy via the bidirectional diagnosis and therapy device in accordance with suggestion and guidance of application software so as to enhance the effect of acupuncture.

The present invention is a smart equipment with bidirectional diagnosis and therapy device, comprising a power supply unit, a high-voltage diagnosis and therapy unit, an input and display unit, a wireless transmission unit, a magnetic disk installed with program and a microprocessor. In this case, the power supply unit is used for converting an external power supply into a required voltage to be supplied for each unit as required power. The input and display unit is provided for inputting operation commands and displaying related image, while the wireless transmission unit is provided for connecting to a cloud database wirelessly.

The high-voltage diagnosis and therapy unit is provided for diagnosis and therapy as well as electronic acupuncture, and sending back a diagnosis and therapy signal. The high-voltage diagnosis and therapy unit is provided with an electronic oscillation generator, two output terminals and an electrode set. An input voltage to the electronic oscillation generator is used for generating an oscillating current outputted at the two output terminals. The electrode set is provided with two output points connected to the two output terminals, respectively.

The microprocessor is then used for processing related operation, while the microprocessor is used for connecting to the power supply unit, the high-voltage diagnosis and therapy unit, the input and display unit, the wireless transmission unit and the magnetic disk installed with program, as well as controlling the operation in accordance with operation commands inputted from the input and display unit.

Furthermore, the magnetic disk installed with program is provided with application software processing the diagnosis and therapy signal correspondingly and being updated online. After the diagnosis and therapy signal is acquired, application software is then allowed for providing an usage suggestion in accordance with the diagnosis and therapy signal. The high-voltage diagnosis and therapy unit is manipulated for diagnosis and therapy by the user via the input and display unit in accordance with the usage suggestion.

Thereby, the user is allowed to perform diagnosis and therapy correctly via the usage suggestion, and application software may be updated online for providing the usage suggestion in response to the latest medical knowledge at any time, such that diagnosis and therapy in the latest and the most correct manner is allowed for the user to obtain the best diagnostic and therapeutic effect.

### BRIEF DESCRIPTION OF THE DRAWINGS

FIG. 1 is a schematic diagram of reflex zones of acupoints on the dorsum of the hand.

FIG. 2 is a schematic diagram of reflex zones of acupoints on the soles of the feet.

FIG. 3 is a functional block diagram of the present invention.

FIG. 4 is an external view of the structure of the present invention.

FIG. 5 is a functional block diagram of a high-voltage diagnosis and therapy unit of the present invention.

FIG. 6 is a circuit diagram of an electronic oscillation generator of the present invention.

FIG. 7 is a diagram showing an electrode set of another embodied mode of the present invention.

FIG. 8 is a diagram showing an electrode set of a further embodied mode of the present invention.

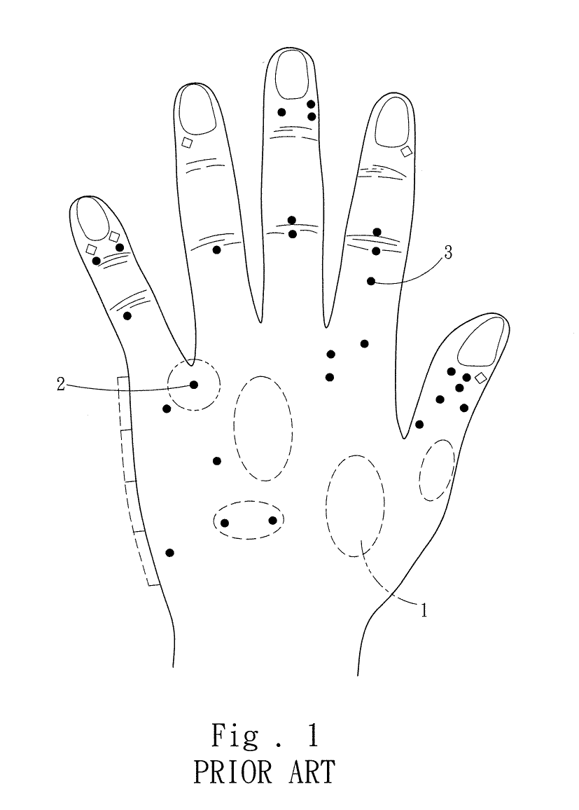

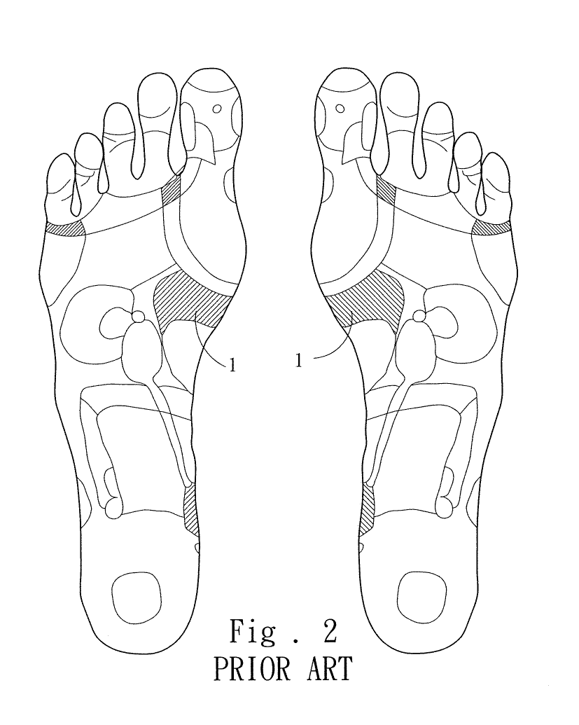

### DETAILED DESCRIPTION OF THE PREFERRED EMBODIMENTS

For better understanding and identifying the features, objects and effects of the present invention, one embodiment will be exemplified and explained in accompany with drawings as follows.

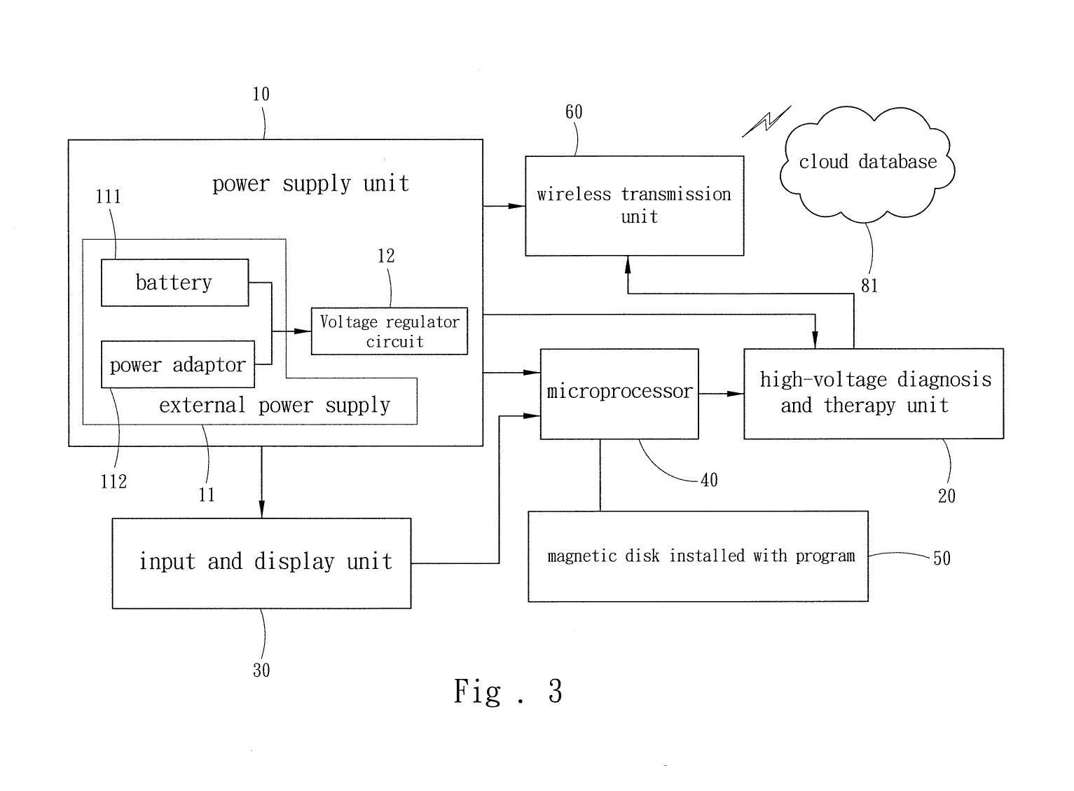

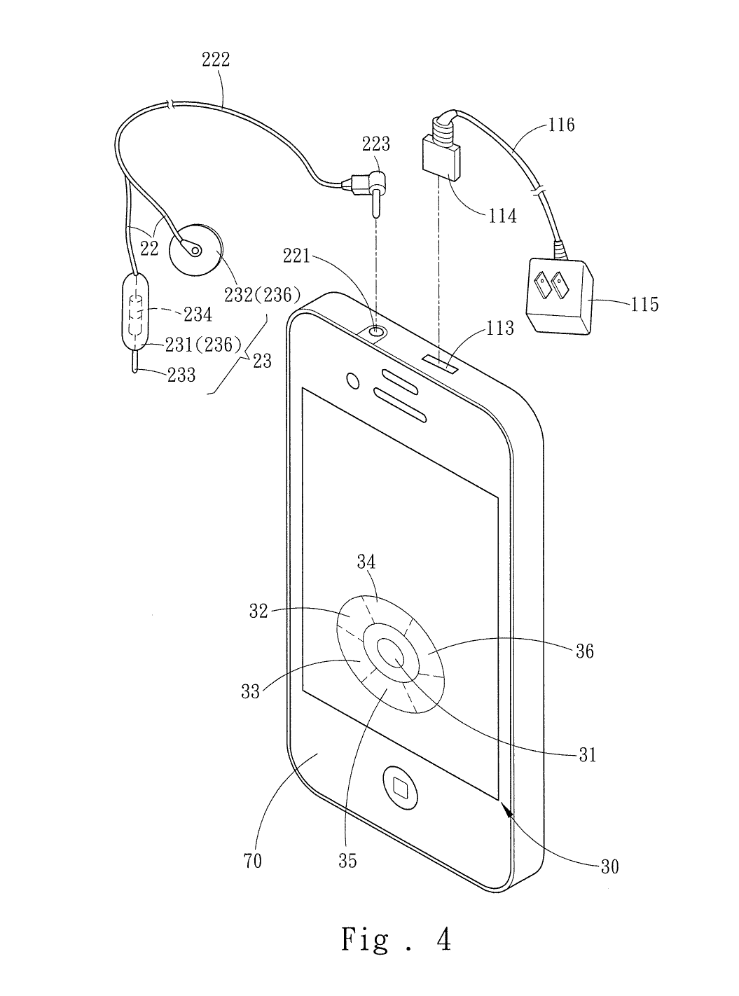

Referring to FIGS. 3 and 4, the present invention is a smart equipment with bidirectional diagnosis and therapy device, in which the smart equipment may be any one of a smartphone and a tablet. The present invention comprises a power supply unit 10, a high-voltage diagnosis and therapy unit 20, an input and display unit 30, a microprocessor 40, a magnetic disk installed with program 50 and a wireless transmission unit 60. In this case, the power supply unit 10, the high-voltage diagnosis and therapy unit 20, the input and display unit 30, the microprocessor 40, the magnetic disk installed with program 50 and the wireless transmission unit 60 are installed in a housing 70.

The power supply unit 10 is used for converting an external power supply 11 into a required voltage to be supplied for each unit as required power. The input and display unit 30 is provided for inputting operation commands and displaying related image to be looked up by a user, while the wireless transmission unit 60 is provided for being connected to a cloud database 81 wirelessly.

The external power supply 11 may be any one of a battery 111 and a power adaptor 112. The battery 111 is portable, while the power adaptor 112 is capable of providing long-lasting power supply. The housing 70 is provided with a power jack 113 electrically connected to a power connector 114, such that power may be acquired from the mains socket (not shown in the figures) via a power cable 116 between the power connector 114 and a mains plug 115. Moreover, for ensuring safety of the circuit, the power supply unit 10 is further provided with a voltage regulator circuit 12 for providing a stable voltage supply.

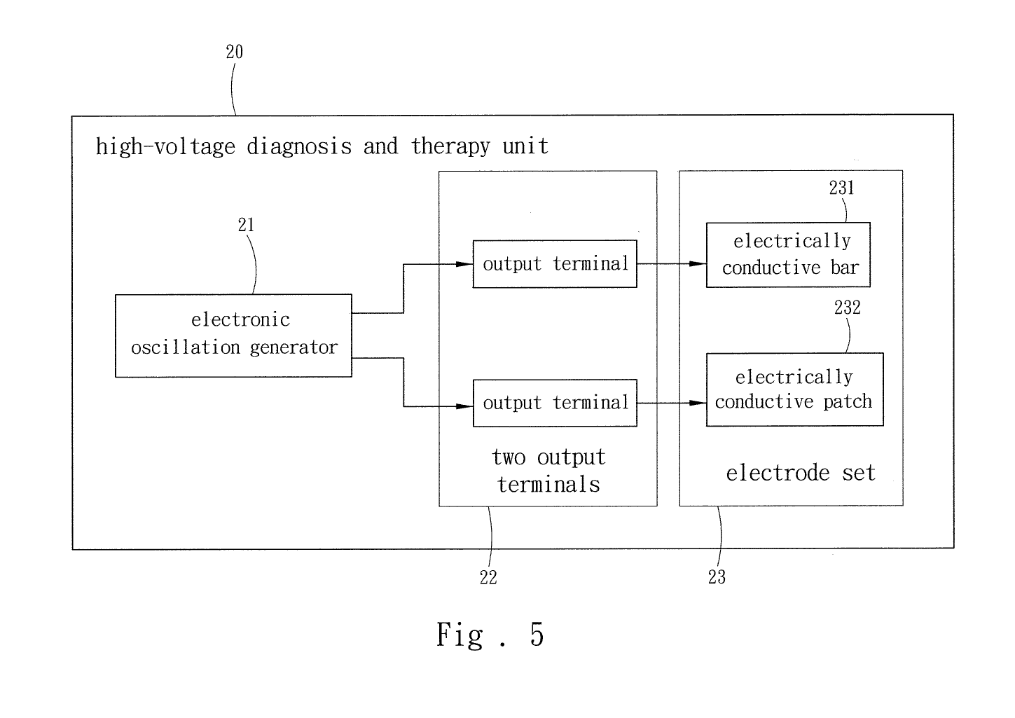

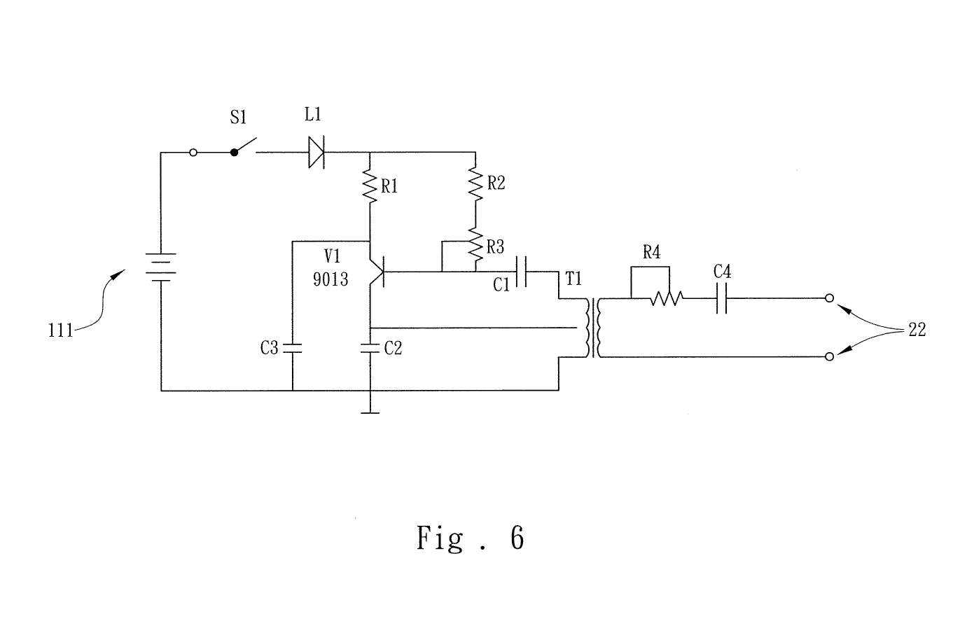

Referring to FIGS. 5 and 6 together, the high-voltage diagnosis and therapy unit 20 is provided with an electronic oscillation generator 21, two output terminals 22 and an electrode set 23. An input voltage to the electronic oscillation generator 21 is used for generating an oscillating current outputted at the two output terminals 22, in which the power source is generated by the battery 111 or the power source is generated by the power adaptor 112 converting alternating current (AC) into direct current (DC). Subsequently, a switch S1 for switching the power source and a light-emitting device L1 indicating whether internal operation is normal are used, followed by generating an oscillating current through a number of resistors R1, R2, variable resistors R3, R4, capacitors C1, C2, C3, C4, and a transistor V1 and transformer T1. Then, the oscillating current is outputted at the two output terminals 22 through the electronic oscillation generator 21.

The electrode set 23 is provided with two output points 236, which may be the combination of an electrically conductive bar 231 and an electrically conductive patch 232 as well as are connected to the two output terminals 22, respectively. The two output terminals 22 are connected to the electrically conductive bar 231 and the electrically conductive patch 232 via an output port 221 on the housing 70, as well as a connector 223 and a Y-shaped connecting wire 222. The leading end of the electrically conductive bar 231 is an electrode 233 made of conductive magnetic material. On the back of the electrode 233, there are provided with a number of magnets 234, by which the current outputted from the output terminal 22 is converted into magnetic wave and then directed to the electrode 233 to be outputted at the leading end of the electrically conductive bar 231.

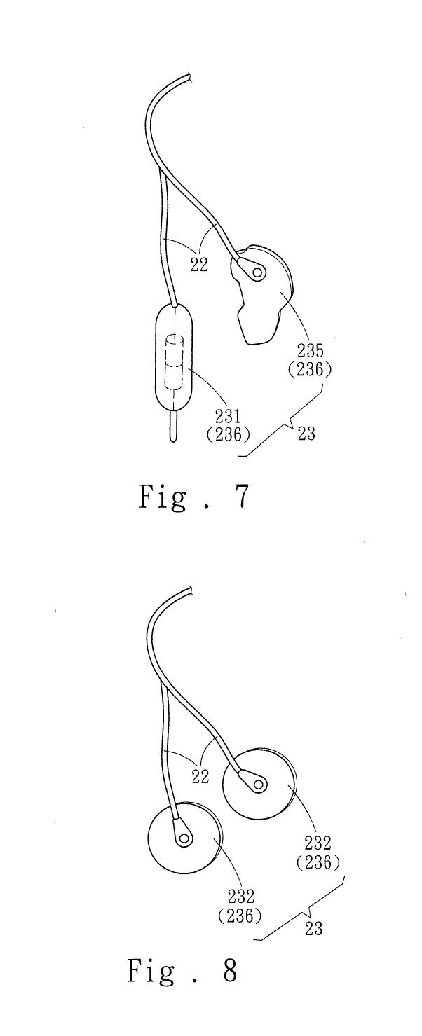

Furthermore, referring to FIGS. 7 and 8, the two output points 236 of the electrode set 23 may be also the combination of the electrically conductive bar 231 and an electrically conductive patch for pinna 235, similarly connected to the two output terminals 22, respectively. The electrically conductive patch for pinna 235 is worn on the ear of human body. The two output points 236 of the electrode set 23, otherwise, may be also the combination of at least two (preferably, 2 to 4) electrically conductive patches 232, which are similarly connected to the two output terminals 22, respectively. The electrically conductive patches 232 are provided for being adhered to human body.

In addition, the input and display unit 30 is generally provided by a touch screen, and virtually provided with a power switch 31, a positive button 32, a negative button 33 and a time-set button 34 for controlling the operation of the high-voltage diagnosis and therapy unit 20. Furthermore, the input and display unit 30 further virtually comprises a diagnosis button 35 and a therapy button 36, which enable the high-voltage diagnosis and therapy unit 20 to provide outputs of specific strengths for diagnosis and therapy, respectively, switched by a user in the pressing manner. It is possible for the user to find out where therapy is required firstly in the diagnostic manner, and then the mode is switched for therapy in the therapeutic manner.

The microprocessor 40 is then used for processing related operation, and connecting to the power supply unit 10, the high-voltage diagnosis and therapy unit 20, the input and display unit 30, the magnetic disk installed with program 50 and the wireless transmission unit 60. The microprocessor 40 is the core of the whole system. The microprocessor 40 is allowed for controlling the operation of various units in accordance with the operation commands inputted from the input and display unit 30, so as to meet individualized requirement of the user.

Furthermore, the wireless transmission unit 60 may be wirelessly connected to a cloud database 81, in which information of diagnosis and therapy may be stored. The information of diagnosis and therapy may be uploaded to the cloud database 81 by the wireless transmission unit 60, and then monitored by specialized medical personnel. Once anxiety about deteriorating health is found, the user may be informed, at the earliest possible time, of further examination at medical institutions. Thus, health status of the user is monitored at any time.

The wireless transmission unit 60 may be a communication element using wireless technology, such as WIFI, Bluetooth and etc., while the magnetic disk installed with program 50 is loaded with application software processing the diagnosis and therapy signal correspondingly as well as being updated online. After the diagnosis and therapy signal is acquired, application software is then allowed for providing an usage suggestion in accordance with the diagnosis and therapy signal. The high-voltage diagnosis and therapy unit 20 is manipulated for diagnosis and therapy by the user via the input and display unit 30 in accordance with the usage suggestion. Application software of the present invention is installed in the magnetic disk installed with program 50, and thus, may be updated to the latest version at any time. Therefore, the usage suggestion received by the user is the latest and the most correct one. Furthermore, in addition to finding out where therapy is required in the diagnostic manner in the present invention, the usage suggestion is further provided through application software-based processing in accordance with the past medical history and physical condition of the user. The best therapeutic effect may be achieved when the mode is switched to a therapeutic mode for therapy by the user, because the usage suggestion is made with reference to the diagnostic result as well as the past medical history and physical condition of the user.

As described above, the present invention enables the user to perform diagnosis and therapy correctly via the usage suggestion made by application software installed in the smartphone. Therefore, application software may be updated online immediately, and then, allowed for providing the usage suggestion in response to the latest medical knowledge at any time. Thus, diagnosis and therapy in the latest and the most correct manner is allowed for the user to obtain the best diagnostic and therapeutic effect.

To sum up, the forgoing is only a preferred embodiment of the present invention, and not used for limiting the scope of the present invention. That is to say, various variations and modifications made in accordance with the patent claims should fall within the technical scope of the present invention.

What is claimed is:

1. A smart equipment with bidirectional diagnosis and therapy device, comprising:
a power supply unit, converting an external power supply into a required voltage;
a high-voltage diagnosis and therapy unit, provided for diagnosis and therapy as well as electronic acupuncture, and sending back a diagnosis and therapy signal, said high-voltage diagnosis and therapy unit being provided with an electronic oscillation generator, two output terminals and an electrode set, an input voltage to said electronic oscillation generator being used for generating an oscillating current outputted at said two output terminals, said electrode set being provided with two output points, said two output points being connected to said two output terminals, respectively;
an input and display unit, provided for inputting operation commands and displaying related image;
a wireless transmission unit, provided for connecting to a cloud database wirelessly;
a magnetic disk installed with program, loaded with an application software processing said diagnosis and therapy signal correspondingly as well as being updated online, said application software being then allowed for providing an usage suggestion in accordance with said diagnosis and therapy signal after said diagnosis and therapy signal is acquired; and
a microprocessor, used for processing related operation, said microprocessor being connected to said power supply unit, said high-voltage diagnosis and therapy unit, said input and display unit, said wireless transmission unit and said magnetic disk installed with program, as well as being allowed for controlling operation in accordance with said operation commands inputted from said input and display unit.

2. The smart equipment with bidirectional diagnosis and therapy device according to claim 1, wherein said external power supply is any one of a battery and a power adaptor.

3. The smart equipment with bidirectional diagnosis and therapy device according to claim 1, wherein said power supply unit is further provided with a voltage regulator circuit.

4. The smart equipment with bidirectional diagnosis and therapy device according to claim 1, wherein said input and display unit is a touch screen, and virtually provided with a power switch, a positive button, a negative button and a time-set button.

5. The smart equipment with bidirectional diagnosis and therapy device according to claim 1, wherein said input and display unit further virtually comprises a diagnosis button and a therapy button, which enable said high-voltage diagnosis and therapy unit to provide outputs of specific strengths for diagnosis and therapy, respectively.

6. The smart equipment with bidirectional diagnosis and therapy device according to claim 1, wherein said two output points of said electrode set are presented as any one of combinations comprising an electrically conductive bar with an electrically conductive patch, said electrically conductive bar with an electrically conductive patch for pinna, and at least two electrically conductive patches, the leading end of said electrically conductive bar being an electrode made of conductive magnetic material, the back of said electrode being provided with a plurality of magnets, by which said current outputted from said output terminals is converted into magnetic wave and then directed to said electrode.

7. The smart equipment with bidirectional diagnosis and therapy device according to claim 1, wherein said power supply unit, said high-voltage diagnosis and therapy unit, said input and display unit, said microprocessor, said magnetic disk installed with program and said wireless transmission unit are installed in a housing.

8. The smart equipment with bidirectional diagnosis and therapy device according to claim 1, wherein said smart equipment is any one of a smartphone and a tablet.
